(19) United States
(12) Patent Application Publication
(10) Pub. No.: US 2006/0258981 A1
(43) Pub. Date: Nov. 16, 2006

(54) Balloon catheter with perfusion lumen

(76) Inventor: Tracee Eidenschink, Wayzata, MN (US)

Correspondence Address: FISH & RICHARDSON PC, P.O. BOX 1022, MINNEAPOLIS, MN, 55440-1022, US

(21) Appl. No.: 11/115,653

(22) Filed: Apr. 27, 2005

(51) Int. Cl. A61M 29/00

(52) U.S. Cl. 604/103.1; 604/916

(57) ABSTRACT

A catheter including an elongate member sized for insertion within a body vessel and having a perfusion lumen extending through the elongate member to at least one distal exit opening and an expandable balloon disposed about a distal portion of the elongate member is described. A method of performing a balloon catheter procedure is described. A system for performing a balloon catheter procedure is also described

### TECHNICAL FIELD

This invention relates to medical devices, and more particularly to perfusing catheters.

### BACKGROUND

Myocardial ischemia (MI), and in severe cases acute myocardial infarction (AMI), can occur when there is inadequate blood circulation to the myocardium due to coronary artery disease. Additionally, the flow of oxygenated blood through the coronary arteries may be reduced or completely blocked by a thrombus or an embolus, and may also be associated with an underlying narrowing of the artery. A full or partial blockage is commonly referred to as a lesion. This full or partial restriction may also lead to an acute myocardial infarction (AMI). Injury to the tissue region continues throughout an ischemic event, as the region is deprived of oxygenated blood. Thus, early treatment of the coronary blockage using, for example, percutaneous transluminal coronary angioplasty (PTCA) or lytic therapy is desirable. Once the lesion in the coronary artery is repaired, normal blood flow may be restored to the ischemic tissue region.

Balloon catheters are used to perform various medical procedures within the body, such as a PTCA. As the balloon catheter is guided through the body to a target location, and the target location often has a very small opening, the distal end of the balloon should have a small cross-sectional diameter (crossing profile). One portion of the crossing profile is the diameter of the catheter's shaft, which must be large enough to allow a guide wire to pass freely through a guide wire lumen in the shaft in a longitudinal direction. Additionally, it is important to have a small crossing profile in the balloon portion of the balloon catheter, as this must pass the area of the lesion. In order to have a smaller crossing profile, catheters typically use guide wires that have cross-sectional diameters in the range of fourteen thousandths of an inch (0.014) up to 35 thousandths of an inch (0.035), with many cardiac procedures performed using balloon catheters with a 0.014 inch guide wire.

Evidence suggests that early reperfusion of blood into the heart, after removing a blockage to blood flow, dramatically reduces damage to the ischemic tissue region and to the myocardium. However, the reestablishment of blood flow may cause a reperfusion injury to occur. It is possible to reduce reperfusion injury by cooling the affected region prior to reperfusion. One method of cooling myocardial tissue is to place an ice pack over the patient's heart. Another method involves puncturing the pericardium and providing cooled fluid to a reservoir inserted into the pericardial space near the targeted myocardial tissue. Cooling of the myocardial tissue may also be accomplished by perfusing the target tissue with cooled solutions, such as by supplying cooled fluid to the target tissue through a catheter placed in the patient's blood vessel. However, introducing a fluid may create a risk of additional perfusion damage caused by supplying a fluid to an area in a body vessel due to fluid pressure and flow.

### SUMMARY

The apparatus and methods illustrated and discussed herein enable the treatment of a target area by perfusing fluids in a manner that decreases risks associated with perfusion of a vessel. If desired, this reduced risk perfusion may be conducted before, during, and after other treatments, such as PTCA, using the same catheter. Thus, the time required for multiple tasks is minimized, and the trauma associated with multiple insertions of different catheters is decreased.

In one aspect, a catheter including an elongate member sized for insertion within a body vessel and having a perfusion lumen extending through the elongate member to at least one distal exit opening, wherein the elongate member includes a distal portion structured so that, during perfusion, the perfusion lumen has, adjacent the at least one distal exit opening, an expanded diameter portion that decreases pressure of perfusion fluid exiting the at least one distal exit opening, and an expandable balloon disposed about a distal portion of the elongate member.

In another aspect, a method of performing a balloon catheter procedure, the method including providing a balloon catheter into a body vessel and to a target area within the vessel, the catheter comprising an elongate member and having a perfusion lumen extending through the elongate member to at least one distal exit opening, wherein the elongate member includes a distal portion structured so that, during perfusion, the perfusion lumen has, adjacent the at least one distal exit opening, an expanded diameter portion that decreases pressure of perfusion fluid exiting the at least one distal exit opening, the catheter further comprising an expandable balloon disposed about a distal portion of the elongate member, and passing fluid through the perfusion lumen and out of the at least one distal exit opening.

In another aspect, a system for performing a balloon catheter procedure, the system including a catheter including a) an elongate member and having a perfusion lumen extending through the elongate member to at least one distal exit opening, wherein the elongate member includes a distal portion structured so that, during perfusion, the perfusion lumen has, adjacent the at least one distal exit opening, an expanded diameter portion that decreases pressure of perfusion fluid exiting the at least one distal exit opening, and b) an expandable balloon disposed about a distal portion of the elongate member, and a control system that controls the delivery of fluid into the catheter perfusion lumen.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a side cross-sectional view, in a longitudinal plane, of an embodiment of a perfusing catheter, with a balloon that is inflated.

FIG. 1B is a side cross-sectional view of the catheter shown in FIG. 1A, with the balloon uninflated.

FIG. 2 is a cross section of the shaft along the line 2-2 shown in FIGS. 1A and 1B.

FIG. 3 is an expanded side cross-sectional view of an alternative embodiment of a catheter.

FIG. 4 is a side cross-sectional view of an alternative embodiment of a catheter.

FIG. 5 is a side cross-sectional view of an alternative adapter for use with various catheter embodiments of the current invention.

FIG. 6 is a cross-sectional view of an alternate shaft for various catheter embodiments.

FIG. 7 is a cross-sectional diagram of a side view of a proximal end of a catheter in one embodiment positioned in a coronary artery, and illustrates a method of treating a target tissue region near the heart.

FIG. 8 is a diagram in side view of a proximal end of a catheter and adapter, and a control system connected to the adapter, with the control system shown in block diagram.

Like reference symbols in the various drawings indicate like elements.

### DETAILED DESCRIPTION

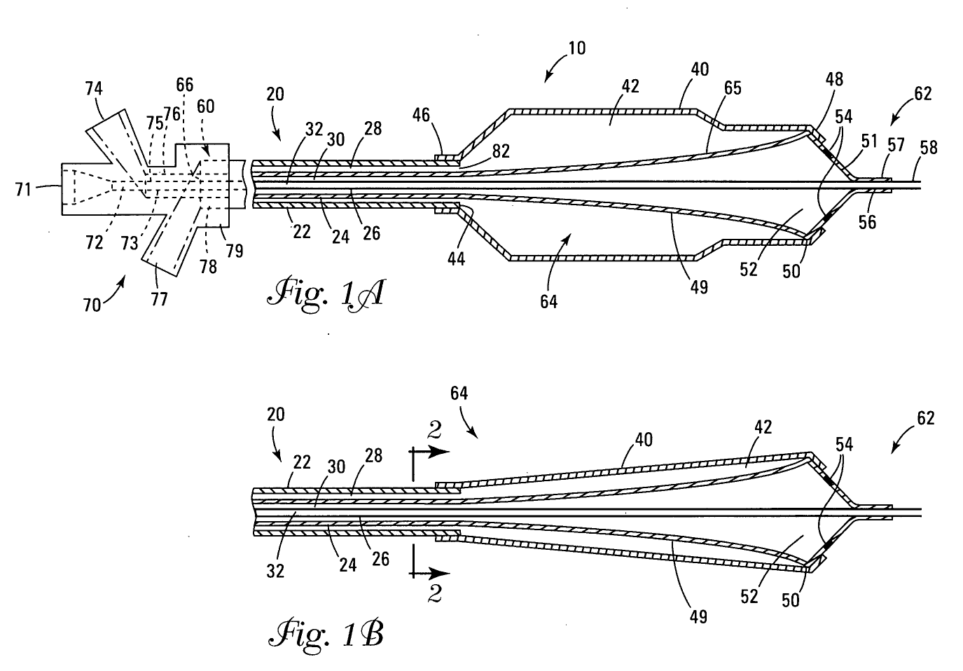

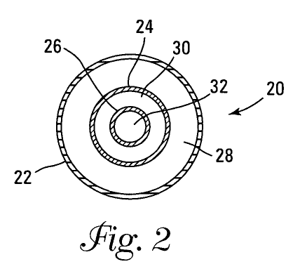

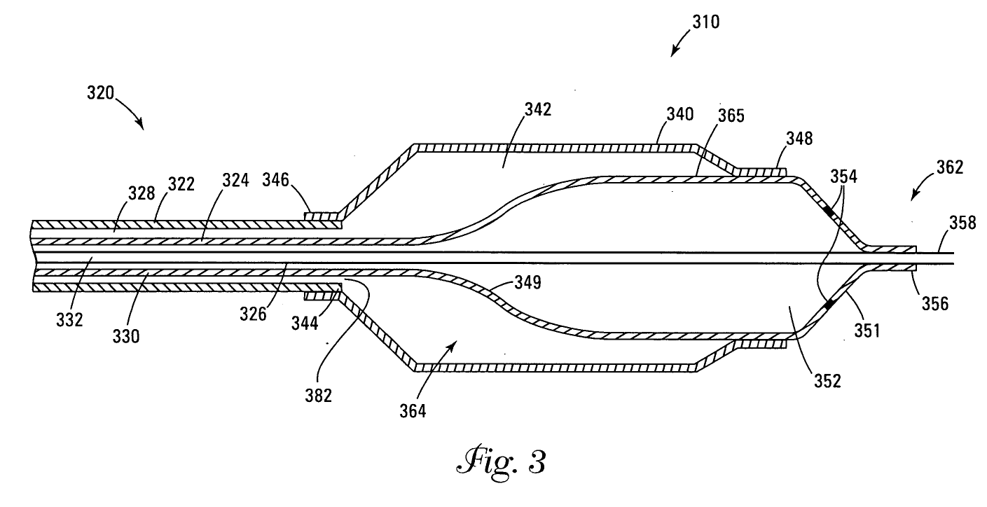

An embodiment of a balloon catheter 10 in accordance with the invention, shown in FIGS. 1A, 1B and 2, includes an elongate shaft 20 with a proximal end 60 and a distal end 62. The catheter 10 also includes a balloon 40 that is formed near the shaft distal end 62 and that encompasses a distal portion 64 of the shaft 20. An adapter 70, shown in FIG. 1A, is shown mated with the catheter 10 at the catheter shaft proximal end 60.

The shaft 20 has a perfusion lumen 30 that extends longitudinally through the entire length of the shaft 20. The lumen 30 extends from an entry port 66 at the shaft proximal end 60 to exit ports 54 (two of which are shown in FIGS. 1A and 1B) near the shaft distal end 62. The perfusion lumen 30 has an expanded diameter portion 52 located near the shaft distal end 62 and in particular adjacent to the lumen exit ports 54. In this implementation, the expanded diameter portion 52 of the perfusion lumen 30 is contained almost entirely within the balloon 40. The expanded diameter portion 52 of the perfusion lumen 30 serves to reduce the local pressure forces near the lumen exit ports 54 so that fluid exiting the ports 54 has a decreased risk of damaging vessel tissue. The use of an expanded area for fluid passage into the body allows a fluid to be provided at higher infusion rates without producing a jetting effect in the body. Jetting occurs when fluid is expelled from the catheter at flow rates that are likely to cause damage to vessel walls and other body tissue. The flow rate of the fluid (Q) is approximately equal to the velocity of the fluid (V) multiplied by the area of the exit location (A), Q=V*A. Therefore, increasing the area through which the fluid passes enables the same fluid flow rate to be achieved while using a lower velocity.

In more detail, the shaft 20 in this implementation includes three elongate tubes, namely, an outer tube 22, an intermediate tube 24, and an inner tube 26. The outer tube 22 extends distally from the shaft proximal end 60 to an outer tube distal end 44 that is affixed with a proximal end 46 of the balloon 40. The intermediate tube 24 extends distally from the shaft proximal end 60 and is contained within the outer tube 22, as can be seen in FIG. 2. A balloon inflation lumen 28 is formed between the outer tube 22 and the intermediate tube 24 between the shaft proximal end 60 and the outer tube distal end 44.

Referring again to FIGS. 1A and 1B, the intermediate tube 24 has a distal portion 65 that extends distally beyond the distal end 44 of the outer tube 22, and through the entire length of the balloon 40 to an intermediate tube distal end 57 that extends distal of the balloon 40.

The inner tube 26 extends distally from the shaft proximal end 60 and is contained within the intermediate tube 24, as can be seen in FIG. 2. Referring again to FIGS. 1A and 1B, the inner tube 26 extends distally through the entire length of the intermediate tube 24 to an inner tube distal end 58 that extends distal of the intermediate tube distal end 57. The perfusion lumen 30 is formed between the intermediate tube 24 and the inner tube 26. A guidewire lumen 32 is formed by the inner tube 26.

Beyond the distal end 44 of the outer tube 22, the intermediate tube 24 gradually flares outward to form the expanded diameter portion 52 of the perfusion lumen 30. This outward-flaring portion 49 of the intermediate tube 24 flares outward to a point 50 of maximum diameter for the expanded diameter portion 52, and from the maximum diameter point 50 an inward-flaring portion 51 flares inward to contact the inner tube 26 distal of the balloon 40. From this point of contact between the intermediate tube 24 and the inner tube 26, the intermediate tube extends distally along the inner tube 26 for a short distance to the intermediate tube distal end 57. An inner surface 56 near the intermediate tube distal end 57 is affixed to an outer surface of the inner tube 26 along this distance located near the distal end 58 of the inner tube 26.

The perfusion exit ports 54 are located on the inward flaring portion 51 of the intermediate tube 24. The perfusion exit ports 54 are located distal of the balloon 40. In FIGS. 1A and 1B, two perfusion exit ports 54 are shown. The intermediate tube distal portion 65 may be made of a flexible material. As such, fluid passing through the perfusion lumen 30 causes the increased diameter portion 52 of the perfusion lumen 30 to expand from a collapsed state (not shown in FIGS. 1A and 1B) to an expanded state shown in FIGS. 1A and 1B. The perfusion exit ports 54 allow fluid to pass from the increased diameter portion 52 of the perfusion lumen 30 out into the body or into a vessel.

As previously mentioned, the balloon 40 is formed near the shaft distal end 62 and encompasses a distal portion 65 of the shaft 20. In particular in the FIGS. 1A and 1B implementation, the balloon 40 is a generally tubular construction having a proximal longitudinal end 46 and a distal longitudinal end 48. An inner surface of the balloon proximal end 46 is affixed to an outer surface of the shaft outer tube distal end 44. In addition, an inner surface of the balloon distal end 48 is affixed to an outer surface of the intermediate tube distal portion 65, and in particular in this implementation, along a small portion of the intermediate distal portion 65 that includes the maximum-diameter point 50 and a portion of the inward-flaring portion 51. The balloon 40 being so affixed to the shaft 20 forms a balloon internal chamber 42 that is bounded by an inner surface of the balloon 40 and an outer surface of the intermediate tube 24.

The previously mentioned balloon inflation lumen 28, which extends distally from its entry port 66 at the shaft proximal end 60, has a port 82 into the balloon internal chamber 42. The balloon 40 may be inflated and deflated while the increased diameter portion 52 of the perfusion lumen 30 is either expanded or unexpanded. FIG. 1B shows the balloon 40 in a deflated state. The balloon 40 may be inflated and deflated by passing inflation medium (gas or liquid) through the balloon inflation lumen 28 and into the balloon internal chamber 42. FIG. 1A shows the balloon 40 in an inflated state. In the FIGS. 1A-1B implementation, the balloon 40, when fully inflated, flares outwardly over a short distance from the proximal end of the balloon 46 to the greatest diameter of the balloon 40. The balloon 40 maintains the same diameter for about 67% of the balloon length, after which the balloon 40 flares inward slightly to a lesser diameter. The balloon 40 maintains this lesser diameter for about 25% of the balloon length, and then flares inward to the balloon distal end 48, which is bonded to a portion of the outer surface of the intermediate tube distal portion 65, as previously described.

The adapter 70 shown in FIG. 1A has three ports, one of which is an inflation port 77 that extends from a side of the adapter 70. The inflation port 77 leads to an adapter inflation lumen 78, which in turn provides access to the catheter inflation lumen 28 when the adapter 70 is mated with the catheter 10. The adapter inflation lumen 78 is formed between an adapter body 79 and an adapter intermediate tube 76.

The adapter 70 also has a perfusion port 74 that extend from the opposite side of the adapter 70 from the inflation port 77. The perfusion port 74 leads to an adapter perfusion lumen 75, which in turn provides access to the catheter perfusion lumen 30. The adapter perfusion lumen 75 is formed between an adapter body 79 and an adapter inner tube 73, and then between adapter intermediate tube 76 and adapter inner tube 73.

The adapter 70 also has a longitudinally located guidewire port 71 that leads to an adapter guidewire lumen 72 that extends longitudinally through the adapter 70. The adapter guidewire lumen 72 in turn provides access to the catheter guidewire lumen 32. The adapter guidewire lumen 72 is formed by the adapter body 79, and an adapter inner tube.

When the catheter shaft proximal end 60 is inserted into the adapter 70 to mate the two components, the adapter body 79 mates with the outer tube 22 of the shaft 20, the adapter intermediate tube 76 mates with intermediate tube 24, and the adapter inner tube 73 mates with inner tube 26. Thus, the adapter inflation port 77 provides access to the catheter inflation lumen 28. Inflation medium (liquid or gas) may be provided to the inflation port 77. The inflation medium then passes into and through catheter inflation lumen 28, and into the balloon internal chamber 42, thereby causing the balloon 40 to inflate. When desired, the balloon 40 can be deflated by removing inflation medium from internal chamber 42 through the inflation lumen 28 and out of port 77. Similarly, the perfusion port 74 provides access to the catheter perfusion lumen 30. Fluid may be provided to the perfusion port 74. The fluid then passes into and through the catheter perfusion lumen 30, into the increased diameter portion 52 of the intermediate tube 24, and then out through the perfusion exit ports 54 in the intermediate tube distal end 51. In like fashion, adapter guidewire port 71 provides access to the catheter guidewire lumen 32, allowing the catheter 10 to be advanced over a guidewire (not shown in FIGS. 1A-1B) by passing the catheter 10 over the guidewire through the guidewire lumen 32. In addition, a guidewire (not shown) may be retracted through the catheter 10, and out through guidewire port 71.

FIG. 3 shows an alternate embodiment of the balloon catheter, and the cross-section is also increased in size so that various aspects may more clearly be seen. FIG. 3 shows a portion of catheter 310, including a portion of elongate shaft 320, and a balloon 340 that is formed near the shaft distal end 362 and that encompasses a distal portion 364 of the shaft 320.

The shaft 320 is similar to the shaft 20 in FIGS. 1A, 1B, and 2. In more detail, the shaft 320 in this implementation includes three elongate tubes, namely, an outer tube 322, an intermediate tube 324, and an inner tube 326. The outer tube 322 extends to an outer tube distal end 344 that is affixed with a proximal end 346 of the balloon 340. The intermediate tube 324 is contained within the outer tube 322. A balloon inflation lumen 328 is formed between the outer tube 322 and the intermediate tube 324 through the shaft 320 to the distal end of the outer tube 344. The inner tube 326 extends through the shaft 320, and is contained within the intermediate tube 324. The perfusion lumen 330 is formed between the intermediate tube 324 and the inner tube 326. A guidewire lumen 332 is formed by the inner tube 326.

Distal of the distal end 344 of the outer tube 322, the intermediate tube 324 rapidly flares outward to form the expanded diameter portion 352 of the perfusion lumen 330. This outward-flaring portion 349 of the intermediate tube 324 flares outward to a maximum diameter 365 for the expanded diameter portion 352. The maximum diameter portion extends distally for more than half the length of the balloon 342 to an inward-flaring portion 351, which flares inward to contact the inner tube 326 distal of the balloon 340. From this point of contact between the intermediate tube 324 and the inner tube 326, the intermediate tube extends distally along the inner tube 326 for a short distance. An inner surface of the intermediate tube prior to the distal end 356 is affixed to an outer surface of the inner tube 326 along a distance located near the distal end 358 of the inner tube 326.

The perfusion exit ports 354 are located on the inward flaring portion 351 of the intermediate tube 324. The perfusion exit ports 354 are located distal of the balloon 340.

The previously mentioned balloon inflation lumen 328 has a port 382 into the balloon internal chamber 342. The balloon 340 may be inflated and deflated while the increased diameter portion 352 of the perfusion lumen 330 is either expanded or unexpanded. The balloon 340 may be inflated and deflated by passing inflation medium (gas or liquid) through the balloon inflation lumen 328 and into the balloon internal chamber 342. FIG. 3 shows the balloon 340 in an inflated state. In the FIG. 3 implementation, the balloon 340, when fully inflated, flares outwardly over a short distance from the proximal end of the balloon 346 to the greatest diameter of the balloon 340. The balloon 340 maintains the same diameter for about 67% of the balloon length, after which the balloon 340 flares inward to the balloon distal end 348. A portion of the balloon 340 up to the balloon distal end 348 is bonded to a length of the intermediate tube 324 along the portion of maximum diameter 365 prior to the inwardly-flaring portion 351 of the intermediate tube 324.

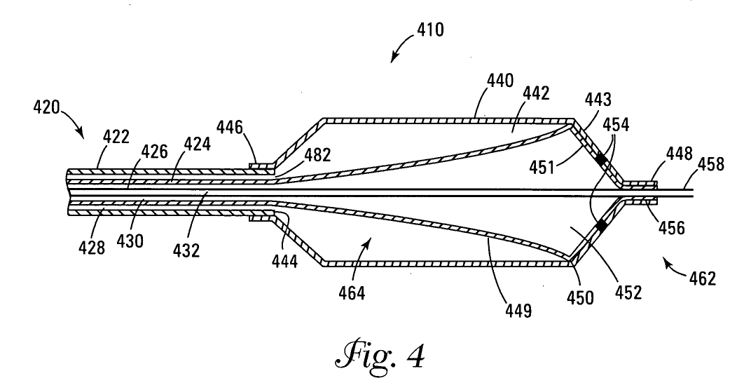

FIG. 4 is yet another embodiment of the balloon catheter, and shows a portion of catheter 410, including an portion of elongate shaft 420, and a balloon 440 that is formed near the shaft distal end 462 and that encompasses a distal portion 464 of the shaft 420.

The shaft 420 is similar to the shaft 20 in FIGS. 1A, 1B, and 2. In more detail, the shaft 420 in this implementation includes three elongate tubes, namely, an outer tube 422, an intermediate tube 424, and an inner tube 426. The outer tube 422 extends to an outer tube distal end 444 that is affixed with a proximal end 446 of the balloon 440. The intermediate tube 424 is contained within the outer tube 422. A balloon inflation lumen 428 is formed between the outer tube 422 and the intermediate tube 424 through the shaft 420 to the distal end of the outer tube 444. The inner tube 426 extends through the shaft 420, and is contained within the intermediate tube 424. The perfusion lumen 430 is formed between the intermediate tube 424 and the inner tube 426. A guidewire lumen 432 is formed by the inner tube 426.

Distal of the distal end 444 of the outer tube 422, the intermediate tube 424 gradually flares outward to form the expanded diameter portion 452 of the perfusion lumen 430. This outward-flaring portion 449 of the intermediate tube 424 flares outward to a point of maximum diameter 450 for the expanded diameter portion 452, and from the maximum diameter point 450 an inward-flaring portion 451 flares inward to contact the inner tube 426 distal of the balloon 440. From this point of contact between the intermediate tube 424 and the inner tube 426, the intermediate tube extends distally along the inner tube 426 for a short distance. An inner surface of the intermediate tube near the intermediate tube distal end 456 is affixed to an outer surface of the inner tube 426 along this distance located near the distal end 458 of the inner tube 426.

The previously mentioned balloon inflation lumen 428 has a port 482 into the balloon internal chamber 442. The balloon 440 may be inflated and deflated while the increased diameter portion 452 of the perfusion lumen 430 is either expanded or unexpanded. The balloon 440 may be inflated and deflated by passing inflation medium (gas or liquid) through the balloon inflation lumen 428 and into the balloon internal chamber 442. FIG. 4 shows the balloon 440 in an inflated state. In the FIG. 4 implementation, the balloon 440, when fully inflated, flares outwardly over a short distance from the proximal end of the balloon 446 to the greatest diameter of the balloon 440. The balloon 440 maintains the same diameter for about 75% of the balloon length until the point of contact with the intermediate tube 424 at the point of maximum diameter 450 of the expanded portion 452. From this point of contact between the balloon 440 and the intermediate lumen 424, an inwardly flaring portion 443 of balloon 440 is bonded with the inwardly-flaring portion 451 of the intermediate tube 424. This bonding continues along the inwardly-flaring portion 451 until the inner surface of the balloon distal end 448 is bonded to the outer surface of the distal end 456 of the intermediate tube

The perfusion exit ports 454 are located on the inward flaring portion 451 of the intermediate tube 424. Two perfusion exit ports 454 are shown in FIG. 4. The perfusion exit ports are thus also located along the inwardly-flaring portion 443 of the balloon 440. Therefore, the perfusion exit ports 454 allow the passage of fluid from the expanded portion of the perfusion lumen 452 to pass through both the intermediate tube 424 and the balloon 440.

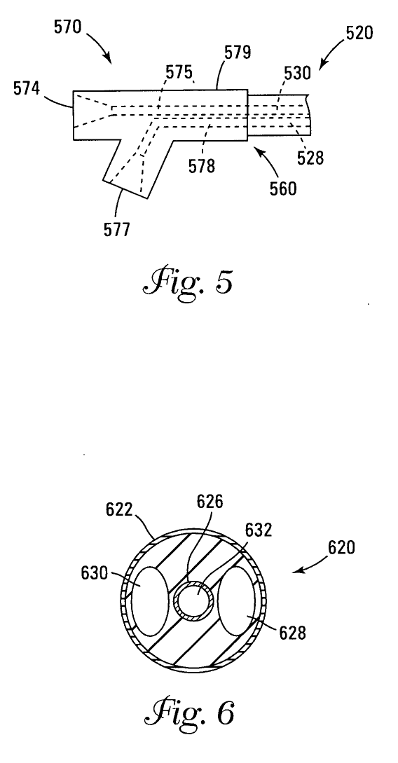

FIG. 5 shows an alternative adapter 570. The adapter 570 has two ports, one of which is an inflation port 577 that extends from the side of the adapter. The inflation port 577 leads to an adapter inflation lumen 578, which in turn provides access to the catheter inflation lumen 528 when the adapter 570 is mated with a catheter shaft 520. The adapter inflation lumen 578 is formed by the adapter body 579.

The adapter 570 also has a longitudinally located perfusion port 574. The perfusion port 574 leads to an adapter perfusion lumen 575, which in turn provides access to the catheter perfusion lumen 530 when the adapter 570 is mated with the catheter shaft 520. The adapter perfusion lumen 575 is also formed by the adapter body 579.

The adapter 570 does not have a guidewire port. Thus, adapter 570 could be used for a fixed-wire (FW) catheter embodiment, as a FW catheter has an embedded guidewire. In addition, adapter 570 could be used for monorail (MR) catheter embodiments, as the guidewire will exit the catheter shaft distally of the proximal end 560 of the catheter 520, before the guidewire reaches the adapter.

When the catheter shaft proximal end 560 is inserted into the adapter 570 to mate the two components, the adapter body 579 mates with the shaft 520. The adapter 570 and shaft 520 are aligned so that the adapter inflation port 577 provides access to the catheter inflation lumen 528, and the perfusion port 574 provides access to the catheter perfusion lumen 530.

FIG. 6 is an cross-section of an alternative shaft that may be used in some embodiments of the invention. Such a shaft might be used for embodiments utilizing an monorail (MR) or fixed-wire (FW) type catheter. A guidewire lumen 632 is formed by an inner tube 626, and runs through the center of the shaft 620. In this embodiment, shaft 620 has an outer tube 622 and is mostly filled, forming the inflation lumen 628 and the perfusion lumen 630. These lumens typically are formed on opposite sides of the guidewire lumen 632.

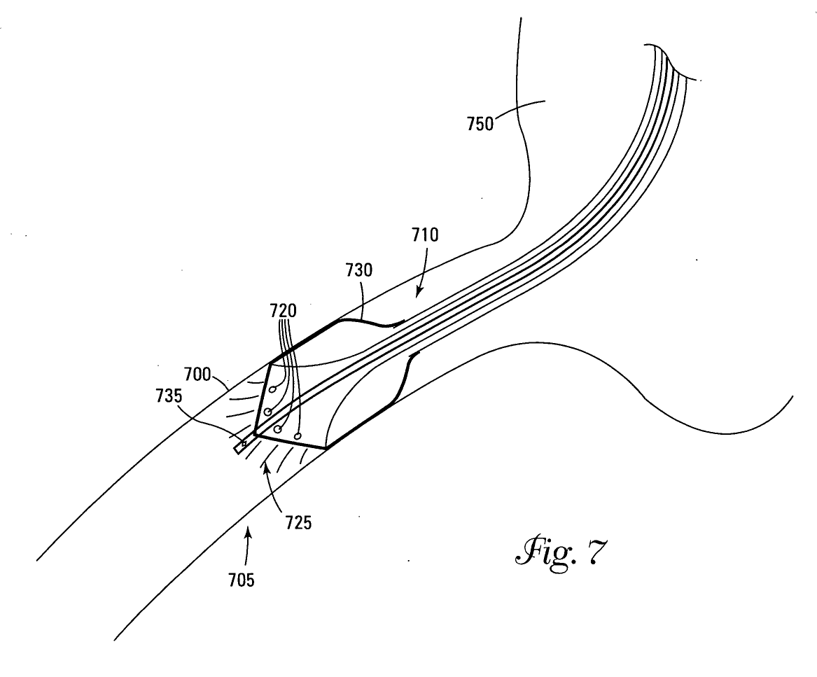

FIG. 7 illustrates a method of treating a target tissue region located in a body vessel. A distal portion 710 of a balloon catheter of the present invention is shown inside a coronary artery 700 near a patient's heart.

The distal portion 710 of the catheter may be positioned inside the coronary artery 700 as shown in FIG. 7 by inserting the catheter's distal end into a vessel, such as a femoral artery, that provides access to a patient's aorta 750. A guidewire (not shown) may be advanced through the aorta 750 and into the desired vessel, which in the FIG. 7 example, is the coronary artery 700. The catheters distal end may then be advanced over the guidewire, through the aorta 750, and into the coronary artery 700.

Once positioned in the coronary artery 700, fluid may be passed through the catheter and out of the perfusion exit ports 720. This introduces a fluid 725 into the target region 705 of the coronary artery 700. The fluid 725 may be cooled in order to cool a target tissue region 705 of the coronary artery 700. Once the tissue region 705 has reached the desired temperature, a balloon 730 may be inflated to treat an area near the target area 705. One such treatment includes using the balloon 730 to treat and repair a lesion in the coronary artery 700 that has reduced or blocked the flow of blood through the vessel. Following treatment of the lesion, the balloon 730 may be deflated, while still continuing to provide fluid 725 to the target region 705. The deflation of the balloon 730 also allows the resumption of blood flow through the coronary artery. After a sufficient period, the flow of fluid 725 may be stopped and the catheter removed.

Optionally, a temperature and/or pressure sensor 735 may be utilized to provide information to the physician about the treatment. The temperature or pressure sensor 735 may be located distally of the balloon 730 as shown in FIG. 7. Alternatively, such a sensor may be located elsewhere, including within the distal portion of the catheter such as that enclosed by the balloon 730.

In other treatments, a balloon 730 may be inflated to occlude the artery 700 and prevent or reduce normal blood flow to the target tissue region 705. In some implementations, the inflation of balloon 730 opens an occlusion in the coronary artery. After normal blood flow has been stopped, a cool fluid 725 may be introduced into the coronary artery to cool the target tissue region 705. Once the target region 705 is cooled to the desired temperature, the balloon 730 may be deflated to resume normal blood flow through the artery 700. To maintain the temperature of the target region 705 within a desired range for an extended period of time, the cooling may be repeated as required. Alternatively, the fluid 725 may be an oxygenated fluid, such as cooled blood, so as to not cause additional oxygen-deprivation damage to the affected area while the cooling is occurring. Such an approach might also be used for a longer period of cooling without depriving the tissue region 705 of oxygen. This may enable the target tissue region 705 to be cooled more rapidly, or to a lower temperature.

Instead of a single inflation step, there may be several iterations of inflation and deflation to treat an effected area. The flow of fluid 725 may be continuous, may be a pre-treatment and/or post-treatment of a balloon or other procedure, or may be stopped and started multiple times. Additionally, the catheter location within the vessel may be adjusted over time.

The treatment methods described may be performed in a vessel that contains a lesion or blockage and is being treated with a percutaneous transluminal coronary angioplasty (PTCA). Alternatively, this method may be performed in a vessel that does not require such treatment. For example, in a procedures where methods are performed in a vessel that does not require the repair of a lesion, cooling fluid may be infused to cool a target tissue region that is near a region where a PTCA is being performed. Additionally, the method may be used to cool organs in the body, such as kidneys, brain, and liver. Alternatively, the method may be used to treat a target area with a drug or pharmaceutical agent, whether alone, or in combination with a cooling treatment.

Additionally, the catheter may be used to cool a target tissue region, including providing cooled fluid or blood to an oxygen-deprived, ischemic tissue region. This cooling may be conducted before, during, after, or independently of the use of the balloon.

The fluid may be provided at different temperatures, volumes, and pressures. This allows a controllable variation in infusion rates. Examples of suitable fluids include blood, cooled blood, saline, cooled saline, or drug doped blood or saline. Examples of suitable drugs include anticoagulants (such as Plavix, Heparin, etc.), PTx, Sirilomus, anti-inflammatories, and anti-rejection drugs.

An inflation medium (gas or liquid) may be used to inflate and deflate the inflatable balloon 730. The inflatable balloon may be a balloon suitable for use in heart vessels, or may be a different balloon for implementation and use in other locations inside the body. The inflation and deflation of the balloon 730 may be controlled manually, such as by a physician or assistant, or in some implementations, may be controlled automatically by a control system located outside of the patient's body, as will be described later.

Fluid flow may be independent of the status of the inflatable balloon 730. Thus, fluid flow may occur before, during, after, or independently of the inflation of the inflatable balloon 730. As the balloon status and fluid flow are independent, pressures may be managed to maintain fluid flow through the perfusion exit ports 720 even when the balloon 730 is inflated. However, there is a point at which the inflation pressure of the inflatable balloon 730 will restrict and stop the fluid flow.

The configuration of the adapter may vary depending on many factors. For example, the access ports may be at different locations, and there may be a variety of different access ports. In addition, the adapter may change based on various catheters that can be used, including an over the wire catheter (OTW), a monorail catheter (MR), or a fixed-wire catheter (FW).

The balloon may have various profiles. For example, the balloon may have a even profile, a stepped profile, or other type of profile.

The expandable area of the intermediate tube may vary in configuration. The distal portion of the intermediate tube may have a gradual increase in diameter to the point of largest diameter, or there may be a rapid increase in diameter to a lengthy region having the largest diameter. Other configurations are also possible.

The distal portion of the intermediate tube may be continuously formed from the same material as the rest of the intermediate tube, or it may be formed of different materials and welded or bonded to the intermediate tube at some point.

The increased diameter portion of the perfusion lumen may have a diameter at its widest point that is 100% or more greater than the diameter of the perfusion lumen in the shaft. In other embodiments, the increased diameter portion may be expanded to have a diameter at its widest point that is 200% or more greater than the diameter of perfusion lumen in the shaft. In other embodiments, the increased diameter portion of the perfusion lumen may be expanded to have a diameter at its widest point that is 300% or more greater than the diameter of perfusion lumen in the shaft. In general, the sum of the area of the perfusion exit ports is greater than the cross-sectional area of the perfusion lumen in the shaft. In some embodiments, the sum of the area of the perfusion exit ports will be at least 1.5 times the cross-sectional area of perfusion lumen in the shaft. In other embodiments, the sum of the area of the perfusion exit ports will be at least 2.0 times the cross-sectional area of perfusion lumen in the shaft.

The perfusion exit ports may be a single area, or may be multiple areas. The area(s) may be open, or may be covered by a mesh, filter, or other partially transmissible material of some type. If there are multiple areas, the areas may be all the same size and shape, or may be different sizes and shapes. The areas may be circular, elliptical, or other shape.

The guidewire lumen may be sized for different possible guidewire diameters. Common cross-sectional diameters of guide wires range from fourteen thousandths of an inch (0.014) up to 35 thousandths of an inch (0.035). The inner tube will preferably have an internal cross section diameter forming a guidewire lumen just slightly larger than the outer diameter of the guidewire (not shown) so that the catheter may be slid easily over the guidewire, but not so much larger to make a significant gap between the guidewire and the inner tube. Commonly, the gap will be from about 0.001 inches to about 0.005 inches.

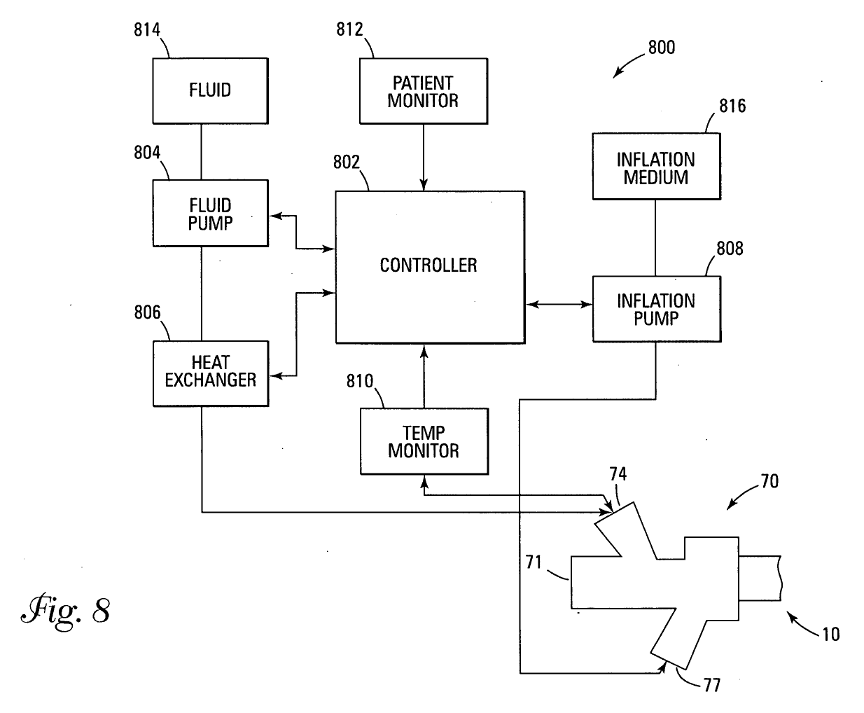

The treatment methods described may be performed manually, or may be performed automatically with the aid of an external control system. FIG. 8 shows a control system along with an adapter 70, such as the adapter 70 of FIG. 1A. In this example, control system 800 includes a controller 802, a fluid pump 804, a heat exchanger 806, an inflation pump 808, an temperature monitor 810, and a patient monitor 812. The controller 802 controls the operation of the fluid pump 804 and the heat exchanger 806, which together dictate the temperature and rate of fluid provided to a target tissue region via the catheter 10. The controller 802 also controls the inflation pump 808, which dictates the inflation of the balloon near the distal end of the catheter. This inflation dictates the amount of normal blood flow to the region by inflating and deflating the inflatable balloon. Additionally, the balloon inflation may be treating a lesion or blockage in a target vessel. Through control of these external devices, the controller 802 may treat a target tissue region for an extended period of time with a fluid.

In FIG. 8, the controller 802 receives input from the other devices in the control system 800 and uses that input, in addition to patient data input manually, or via a link to another system, to coordinate the providing of fluid and the flow of normal blood to the tissue region. For example, the fluid pump 804 provides the controller 802 with the rate at which the fluid 814 is infused through the catheter 10 to the tissue region. The inflation pump 808 provides the controller 802 with the pressure of the balloon and the pressure of the inflation medium 816. Through this information, the controller 802 can determine the extent to which the balloon is inflated or deflated. The patient monitor 812 provides the patient's physiologic data to the controller 802, such as the patient's heart rate, heart rhythm, blood pressure, blood oxygen level, etc. From this information, the controller 802 can provide an alarm or alert the doctor that the patient is experiencing complications, or may adjust the treatment procedure automatically. Optionally, a temperature monitor 810 may be used to provide information about a temperature of a region near the distal end of the catheter. This might be a temperature of the fluid inside the catheter, the fluid as it leaves the catheter, a region near the catheter, or the temperature of a region near the target tissue region. In cases where a temperature monitor 810 is not used, the fluid pump 804 or heat exchanger 806 might provide temperature information about the fluid.

The controller 802 may also receive information about the procedure to be performed, including the specific catheter to be used, a cooling technique to be used, and inflation/deflation plan or schedule for the inflatable balloon, the vessel which will be treated, the type of fluid being applied to the target tissue region, a schedule profile of fluid pressure and perfusion rates, the total length of the procedure, the target temperature range for the fluid, the total volume of fluid to be used, and other information. In some applications, the temperature of the target tissue region cannot be directly measured. This is often true when the temperature cannot be measured without performing a more invasive procedure. In such a case, and if the temperature measurement is desired, the control system 800 may include a second temperature monitor for monitoring the temperature of a target tissue region using another measurement device. For example, a temperature might be calculated using the temperature and infusion rate of the fluid, the flow rate of normal blood, and body temperature of the patient. Many variations of calculated temperatures and alternative monitoring schemes are possible.

The control data may also include procedural constraints, such as the maximum infusion rate of the fluid, the pressure at which the expandable sheath should be maintained, a maximum and minimum temperature for the target tissue region, as well as other possible constraints. With respect to control of the catheter's balloon or other device, the control data may indicate the size of the body vessel and the required pressure to inflate the balloon, or the maximum extent to which another device, such as a cage, may be opened. These are only examples of some of the control data that may be provided to the controller 802 to control a treatment procedure.

After receiving the patient data, inputs from other devices, any manually entered information, and any control data, the controller 802 processes the information in accordance with the control data and provides output to the various components of the control system 800. For example, it provides output to the fluid pump 804, and the heat exchanger 806 to control the infusion rate and temperature of fluid 814. It is able to provide output to the inflation pump 808 to control the inflation or deflation of an inflatable balloon using inflation medium 816. During the procedure, the controller 802 continually monitors the progress of the procedure and adjusts the outputs in accordance with the procedure being performed. The controller 802 may be a digital, analog, or other type of controller.

The fluid 814 used for perfusion may be any biocompatible fluid. The fluid selected will be based upon the purpose of the procedure. The fluid 814 may also contain additives that may be changed during the procedure. These additives may be added to the fluid 814 using a pump not shown in FIG. 8. The fluid 814 may be perfused through the perfusion lumen using a pump 804. For example, a positive displacement pump may be used to provide the necessary pressure to push the fluid through the narrow infusion lumen, and into the expandable sheath. In some implementations, the pump 804 may be replaced by a manual system including a raised bag of fluid, where gravity, or a pressure cuff may be used to control the perfusion rate of the fluid 814. The fluid pump 804 may include a perfusion monitor to monitor the pressure and flow rate of the fluid through the perfusion lumen. It may also include a temperature sensor to monitor the temperature of the fluid.

If the fluid 814 is to be warmed or cooled, a conventional heat exchanger may be used. In one implementation, the heat exchanger 806 is controlled by the controller 802 by processing the information received from temperature sensors and manual input, which may include a temperature monitor 810. Based on the information provided to the controller 802, the heat exchanger 806 may be used to cool or warm the fluid 814 provided to the target tissue region. In implementations where a temperature sensing device is not used to measure the temperature at the target tissue region or inside the catheter, temperature monitor 810 may be excluded or omitted.

The inflation medium 816 may be infused through the inflation lumen of the catheter 10 by a conventional inflation pump 808. The inflation medium may be either a gas or a liquid. In one implementation, the inflation pump 808 is a positive displacement pump. In other implementations, it may be a different kind of pump, such as, for example, a pneumatic pump or a hydraulic pump. In implementation where the catheter's balloon is not inflated as part of the treatment, or in implementations where the catheter does not have an inflatable balloon, the inflation pump 808 and inflation medium 816 may be omitted from the system. As another alternative, the inflation and deflation of an inflatable balloon may be performed manually, and in such a case, the inflation pump 808 would also be omitted from the system.

A number of embodiments of the invention have been described. Nevertheless, it will be understood that various modifications may be made without departing from the spirit and scope of the invention. Accordingly, other embodiments are within the scope of the following claims.

What is claimed is:

1. A catheter comprising:
an elongate member sized for insertion within a body vessel and having a perfusion lumen extending through the elongate member to at least one distal exit opening, wherein the elongate member includes a distal portion structured so that, during perfusion, the perfusion lumen has, adjacent the at least one distal exit opening, an expanded diameter portion that decreases pressure of perfusion fluid exiting the at least one distal exit opening; and
an expandable balloon disposed about a distal portion of the elongate member.

2. The catheter of claim 1, wherein the expandable balloon encompasses at least a portion of the expandable diameter portion of the perfusion lumen.

3. The catheter of claim 1, wherein the expandable balloon is positioned such that fluid exiting the at least one perfusion lumen exit opening exits distal of a longitudinal point of largest diameter of the expandable balloon when in an expanded state.

4. The catheter of claim 1, wherein the expandable balloon is positioned such that fluid exiting the at least one perfusion lumen exit opening exits distal of the expandable balloon.

5. The catheter of claim 1, wherein the elongate member expandable distal portion and the expandable balloon are structured so that the perfusion lumen expanded diameter portion may be formed even while the expandable balloon is in an expanded state.

6. The catheter of claim 1, wherein at least a portion of the elongate member distal portion is made of a material that expands outwardly and forms the expanded diameter portion of the perfusion lumen during a time perfusion is occurring and is collapsible during a time perfusion is not occurring.

7. The catheter of claim 1, wherein the elongate member further has a balloon inflation lumen extending through the elongate member to a port into an internal chamber of the expandable balloon.

8. The catheter of claim 7, wherein the port into the internal chamber of the expandable balloon is located at a proximal end of the expandable balloon internal chamber.

9. The catheter of claim 8, wherein the elongate member and the perfusion lumen formed therein extends distally beyond the port and through the expandable balloon internal chamber.

10. The catheter of claim 7, wherein the expandable balloon has a proximal end that is affixed about a portion of an outer surface of the elongate member that is located immediately proximal of the port from the balloon inflation lumen into the expandable balloon internal chamber.

11. The catheter of claim 10, wherein the expandable balloon has a distal end that is affixed about a portion of an outer surface of the distal portion of the elongate member.

12. The catheter of claim 1, wherein the at least one perfusion lumen distal exit opening has a total combined cross-sectional area that is greater than the cross-sectional area of the perfusion lumen at a location that is proximal of the elongate member distal portion.

13. The catheter of claim 12, wherein the at least one perfusion lumen distal exit opening comprises a plurality of openings.

14. The catheter of claim 12, wherein the elongate member includes a mesh material that forms the at least one perfusion lumen distal exit opening.

15. The catheter of claim 1, wherein the perfusion lumen expanded diameter portion has a diameter that increases gradually, starting from a proximal end of the expanded diameter portion, to a maximum expanded diameter.

16. The catheter of claim 1, wherein the perfusion lumen expanded diameter portion has a diameter that increases abruptly, starting from a proximal end of the expanded diameter portion, to a maximum expanded diameter.

17. A method of performing a balloon catheter procedure, the method comprising:
providing a balloon catheter into a body vessel and to a target area within the vessel, the catheter comprising an elongate member and having a perfusion lumen extending through the elongate member to at least one distal exit opening, wherein the elongate member includes a distal portion structured so that, during perfusion, the perfusion lumen has, adjacent the at least one distal exit opening, an expanded diameter portion that decreases pressure of perfusion fluid exiting the at least one distal exit opening, the catheter further comprising an expandable balloon disposed about a distal portion of the elongate member; and
passing fluid through the perfusion lumen and out of the at least one distal exit opening.

18. The method of claim 17, further comprising inflating the balloon so that the balloon is inflated during a time the fluid is being passed through the perfusion lumen and out of the at least one distal exit opening.

19. The method of claim 17, wherein the fluid is a liquid cooled to a temperature that is below a normal core body temperature.

20. The method of 17, wherein the procedure is a percutaneous transluminal coronary angioplasty procedure.

21. The method of claim 17, further comprising:
providing, before providing the balloon catheter into the body vessel, a guidewire into the body vessel; and
providing the balloon catheter into the body vessel and to the target area within the vessel by passing the catheter over the guidewire.

22. A system for performing a balloon catheter procedure, the system comprising:
a catheter comprising a) an elongate member and having a perfusion lumen extending through the elongate member to at least one distal exit opening, wherein the elongate member includes a distal portion structured so that, during perfusion, the perfusion lumen has, adjacent the at least one distal exit opening, an expanded diameter portion that decreases pressure of perfusion fluid exiting the at least one distal exit opening, and b) an expandable balloon disposed about a distal portion of the elongate member; and
a control system that controls the delivery of fluid into the catheter perfusion lumen.

23. The system of claim 22, wherein:
the catheter elongate member further has a balloon inflation lumen extending through the elongate member to a port into an internal chamber of the expandable balloon; and
the control system further controls providing an inflation medium into and from the inflation lumen to inflate and deflate the expandable balloon.

24. The system of claim 22, wherein:
the expandable catheter balloon encompasses at least a portion of the catheter elongate member distal portion; and
the catheter elongate member distal portion and the expandable balloon are structured so that the perfusion lumen expanded distal portion is formed even while the expandable balloon is in an expanded state.
